(19) United States
(12) Patent Application Publication
(10) Pub. No.: US 2004/0260384 A1
(43) Pub. Date: Dec. 23, 2004

(54) Superelastic coiled stent

(75) Inventor: Jeffrey W. Allen, Santa Rosa, CA (US)

Correspondence Address: Catherine C. Maresh, IP Legal, 3576 Unocal Place, Santa Rosa, CA, 95403, US

(73) Assignee: MEDTRONIC AVE

(21) Appl. No.: 10/463,124

(22) Filed: Jun. 17, 2003

(51) Int. Cl. A61F 2/06

(52) U.S. Cl. 623/1.12; 623/1.22

(57) ABSTRACT

The present invention provides a system for treating an aneurysm, including a catheter with a stent-receiving tube and a coiled stent axially received in the stent-receiving tube, wherein the coiled stent is delivered to the aneurysm through a vessel via the catheter. The present invention also provides a coiled stent and a method of treating an intracranial aneurysm.

### FIELD OF THE INVENTION

This invention relates generally to biomedical stents. More specifically, the invention relates to a superelastic coiled stent for the treatment of intracranial aneurysms.

### BACKGROUND OF THE INVENTION

Aneurysms in the brain and in other parts of the body typically occur due to a weakness of the arteries that allows an abnormal bulge to develop in the arterial wall. A cerebral aneurysm is caused by a weakness in the wall of an artery or vein and often occurs at the junctions of the large arteries at the base of the brain. If an aneurysm bursts, it may cause hemorrhages and stroke-like problems such as paralysis, speech difficulties, memory loss or even death. Bleeding from a cerebral aneurysm is fatal in many cases and those that survive are often disabled.

The primary goal of treatments for cerebral aneurysms is to prevent further enlargements and future ruptures of the endovascular wall. Aneurysms can be treated external to the blood vessel using surgical techniques or from inside the blood vessel using endovascular techniques. Traditionally, surgeons have treated ruptured and unruptured cerebral aneurysms by opening up the skull and excluding or closing the aneurysm with a surgical clip placed at the neck or mouth of the aneurysmal sack close to the blood vessel. In this approach, the affected artery must be exposed and the aneurysm visualized directly. The advent of microneurosurgical techniques and advancements in cerebrovascular surgery such as temporary clipping and neuroprotection have extended the applicability of aneurysm surgery and improved surgical outcomes. In spite of these advancements, there remain aneurysms that are difficult to treat surgically.

In contrast to surgery, an endovascular treatment of an aneurysm may be performed with a catheter similar to that used during an arteriogram. Through the catheter, the cavity of the aneurysm is packed with material that prevents arterial blood to flow into it, a technique referred to as embolization. Materials used for aneurysm embolization include soft and flexible platinum coils with intrinsic helical memory of approximately 100 microns in diameter. The treatment involves transarterial delivery of platinum coils into the lumen of the aneurysm by way of a microcatheter placed into the aneurysm. Once inside the aneurysm, an electric current may be used to detach the end of a coiled wire from the catheter, leaving the coil in place. This procedure is repeated until the aneurysm is largely filled with coils, causing blood flow to bypass the aneurysm. The purpose of the coil is to encourage quick formation of a thrombus or blood clot around the coil.

A conventional vaso-occlusive coil can be made of a helically-wound coil of platinum wire, with a stretch-resistant wire attached within the primary coil between two end caps. Unfortunately, such a construction has a relatively complex and nonlinear bending characteristic, dependent on the spacing of the coils and the radius of the bend of the coil.

Villar et al. describes a helically coiled apparatus in “Vaso-Occlusive Device with Attached Polymeric Materials”, U.S. Pat. No. 6,287,318 issued Sep. 11, 2001. This device comprises a helically-wound metallic core and two polymeric conjuncts of differing thrombogenicity that are woven into a braid. The inventor suggests that the combination of coils with fibrous additions is able to produce neocapillary formation in the vasculature.

Coatings with bioactive agents have been added to coils used for treatment of aneurysms. Coiled vaso-occlusive devices coated with bioactive agents or a collagen material are disclosed by Boock, et al. in “Bioactive Coating for Vaso-Occlusive Devices”, U.S. Pat. No. 6,187,024 issued Feb. 13, 2001.

Medical professionals have estimated that the coil technique is the preferred treatment method for a majority of patients with intracranial aneurysms. Medical trials have indicated that the coil technique may reduce deaths and disability by almost a quarter compared to surgery. This treatment of aneurysms is less invasive than other procedures, allowing for faster recovery time and shorter hospital stays. The coils, however, may be difficult to deliver to distal tortuous vasculature or may not fully or partially exclude an aneurysm. It is important that the coils used to occlude aneurysms remain in place where they are implanted. However, migration of the coils after placement is a problem that may be encountered with these coils. A method using an anchored embolization coil within the intracranial vasculature is disclosed by Berryman, et al. in “Method of Intracranial Vascular Embolotherapy Using Self Anchoring Coils”, U.S. Pat. No. 6,126,672 issued Oct. 3, 2000.

Some aneurysms, such as those at the base of the skull or very large aneurysms, are unsuitable for either surgical management or embolization with platinum coils. Occasionally the closure of a large aneurysm is managed with the least risk when a number of detachable balloons can be placed near the aneurysm. The Federal Drug Administration (FDA) of the United States approved the use of detachable silicone balloons in 1998. This balloon procedure involves a permanent closing of the artery in question and may be used when a patient can tolerate occlusion of the artery. When an occlusion of the artery would prevent required blood flow, a bypass procedure before closure of the vessel may be required.

Other types of devices proposed for use with aneurysms include a flexible patch or bulbous devices with an anchor. An aneurysm patch using a flexible material that is delivered to the mouth of an aneurysm through a vessel is disclosed by Maynard in “Aneurism Patch including Distributed Activator for a Two-Dimensional Shape Memory Alloy”, U.S. Pat. No. 6,409,749 issued Jun. 25, 2002. The patch may be effective in adhering to a saccular aneurysm, also referred to as sacculated or berry aneurysm. Typically, saccular and lateral aneurysms distends only on one side of the vessel, often at areas of bifurcation along a curve of the parent vessel, and point in the direction that the flow would proceed had the curve not been present. The aneurysm patch may be less effective with unusually shaped recesses of an aneurysm or with nonsaccular or axial aneurysms that tend to involve the entire circumference of the blood vessel and cause an otherwise generally cylindrical segment of the vessel to balloon outward.

An aneurysm occlusion device with a bulbous body portion and anchor is disclosed by Mazzocchi in “Method and Apparatus for Occluding Aneurysms”, U.S. Pat. No. 6,168,622 issued on Jan. 2, 2001. The resilient body and anchor of the device may work with an enlarged body and a narrowed neck connecting the body to a wall of the vessel in other parts of a body besides intracranial vessels, but may not be suited for delicate brain tissue and delivery through tortuous brain vessels.

Stents are among the suggested occlusive devices for use in vascular surgery of the brain. Conventional stents are implanted within a vessel in a contracted state and expanded when placed at the desired location in the vessel, in order to restore and maintain patency of the vessel. They are typically deployed by mounting the stent on a balloon portion of a balloon catheter, positioning the stent in a body lumen, and expanding the stent by inflating the balloon. The balloon is then deflated and removed, leaving the stent in place. However, the placement, inflation and deflation of a balloon catheter is a complicated procedure involving risks to delicate intracranial vasculature beyond the implantation of the stent, so that it would be desirable to provide a simpler stent system that places the contracted stent in the site to be treated, and deploys the stent without the use of a balloon. Also, conventional stents are bulky and rigid relative to the delicate vasculature of the brain.

Stents commonly have a metallic structure to provide the strength required to function as a reinforcement structure for a vessel, but typically do not provide for the delivery of localized therapeutic pharmacological treatment of the vessel at the location being treated with the stent. Stents formed from polymeric materials capable of carrying and releasing therapeutic agents may not provide adequate structural and mechanical requirements for a stent, especially when the polymeric materials are loaded with drugs, since drug loading of a polymeric material can significantly affect the structural and mechanical properties of the polymeric material. Since it is frequently desirable to be able to provide localized therapeutic treatment of a vessel at the location being treated with the stent, it would be desirable to combine such polymeric materials with a stent structure to provide the stent with the capability of carrying and delivering therapeutic drugs or other bioactive agents at a specific site in the vasculature to be treated.

A collagen-coated, superelastic stent formed from a tube of collagen with an inner structure of micro-cable is disclosed by Ferrera and Wilson in “Coated Superelastic Stent”, U.S. Pat. No. 6,497,671 issued on Dec. 24, 2002. The helical-shaped stent employs one or more flexible strands of shape memory material disposed within a collagen tube that form a ribbon. The micro-coiled stent may be used with brain aneurysms, entering into the lumen of the aneurysm by way of a microcatheter placed into the aneurysm. This procedure fills the aneurysm with coils of the stent, similar to the aneurysm treatments using microcoils as described above.

Another coiled stent is disclosed by Bosely in “Pull Apart Coil Stent”, U.S. Pat. No. 5,514,176 issued on May 7, 1996. This coiled stent, unlike the previously mentioned stent, is surgically placed rather than being deployed by a catheter and is designed to be placed in the body only temporarily. It is probably a less desirable occlusion device for brain aneurysms where permanent devices are preferred, although it illustrates that closely wound coil loops can be substantially imperforate.

Vaso-occlusive devices and stents have provided important treatments of the vasculature. However, it would be desirable to provide a stent system and method that offers a simpler procedure to occlude an intracranial aneurysm that is less likely to break vessels or damage tender brain tissue, provides an effective treatment for small, potentially lethal aneurysms, and can be used with other coiled procedures.

### SUMMARY OF THE INVENTION

One aspect of the present invention provides a system for treating an aneurysm, including a catheter with a stent-receiving tube and a coiled stent. The coiled stent is axially received in the stent-receiving tube, and delivered to the aneurysm through a vessel via the catheter. The coiled stent forms a helical spring when deployed at the aneurysm.

Another aspect of the present invention is a coiled stent for treating an aneurysm, including a coiled stent framework coaxially located within a stent-receiving tube when axially elongated within the stent-receiving tube, and recoiled when delivered from the stent-receiving tube and deployed adjacent to the aneurysm.

Another aspect of the invention is a method of treating an intracranial aneurysm. A catheter including an axially-received coiled stent is inserted into a vessel in a body. The axially-received coiled stent is positioned adjacent to the intracranial aneurysm and separated from the catheter. At least a portion of the recoiled stent occludes an aneurysmal neck of the intracranial aneurysm when the coiled stent is deployed.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

FIG. 1 is an illustration of a coiled stent deployed at an intracranial aneurysm, in accordance with one embodiment of the current invention;

FIG. 2 is a perspective view of a coiled stent formed as a helical spring, in accordance with one embodiment of the current invention;

FIG. 3 is an illustration of a system for treating an aneurysm, in accordance with one embodiment of the current invention;

FIG. 4 is a perspective view of a coiled stent formed as a helical spring with at least one taper or undulation, in accordance with one embodiment of the current invention;

FIG. 5 is a cross-sectional view of a coiled stent formed as a helical spring with at least one taper or undulation, in accordance with another embodiment of the current invention;

FIG. 6 is a cross-sectional view of a coiled stent with an adhesion layer and a polymeric coating circumferentially disposed along a portion of the coiled stent, in accordance with one embodiment of the current invention; and

FIG. 7 is a flow diagram of a method of treating an intracranial aneurysm, in accordance with one embodiment of the current invention.

### DETAILED DESCRIPTION OF THE PRESENTLY PREFERRED EMBODIMENTS

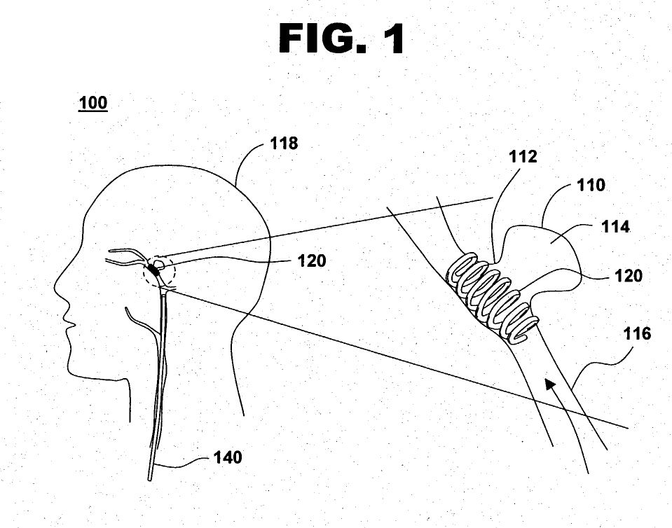

FIG. 1 shows an illustration of a coiled stent deployed at an intracranial aneurysm, in accordance with one embodiment of the present invention at 100. A coiled stent 120 is deployed via a catheter 140 adjacent to an intracranial aneurysm 110.

Coiled stent 120 may be positioned to occlude an aneurysmal neck 112 of intracranial aneurysm 110. Aneurysmal neck 112 is also referred to as an aneurysmal mouth, the region where an aneurysmal sack 114 adjoins an intracranial vessel 116. Aneurysmal neck 112 may have a wide opening, a narrow opening, or an irregularly shaped opening, and is sometimes referred to as the mouth of the aneurysm. Occlusion of an aneurysm prevents or limits the movement of endovascular fluids between intracranial aneurysm 110 and intracranial vessel 116. Intracranial vessel 116 may be one of the many delicate intravasculature vessels within the intracranial vasculature of a cranium 118.

Intracranial aneurysm 110 may be an axial aneurysm, a lateral aneurysm, a saccular aneurysm sometimes referred to as a berry aneurysm, or any aneurysm formed within cranium 118. Although depicted as a treatment method for an intracranial aneurysm, the invention may be used at other suitable locations within the body to treat aneurysms and for other procedures using coiled stents.

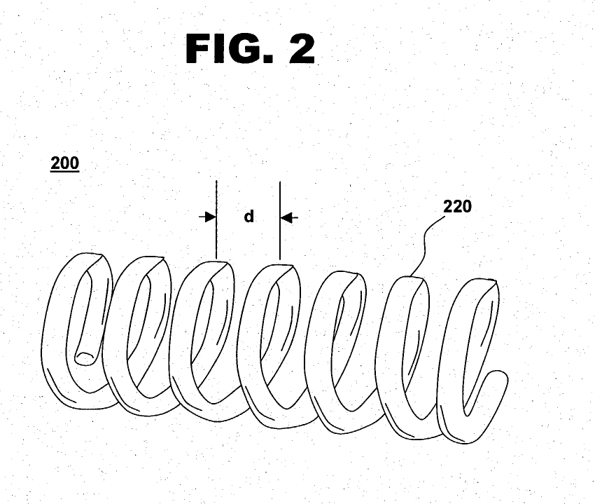

FIG. 2 shows a perspective view of a coiled stent formed as a helical spring, in accordance with one embodiment of the present invention at 200. Coiled stent 220 winds helically about a central axis with a center-to-center spacing d between adjacent windings of the stent.

In one embodiment, the spacing d between adjacent windings, the diameter of wire forming the stent framework, and the outer diameter and inner diameter of coiled stent 220 are nominally constant along an axial length of coiled stent 220. The shape of coiled stent 220 may be set so that there is a small spacing between adjacent turns of the coil. The compliance of the helical spring is determined in part by the diameter of the wire, the number of windings, the material composition, and the outer and inner diameter of coiled stent 220. Coiled stent 220 may be manufactured to form a helical spring when the coiled stent is deployed at the aneurysm. In one example, the diameter of the stent framework wire is less than 0.010 inch in diameter. In another example, the diameter of the stent framework is nominally 0.001 inch in diameter. The coiled stents may have an outer diameter, for example, between one and five millimeters, and a length from a few millimeters to 30 millimeters or longer.

Coiled stent 220 comprises a coiled stent framework. The coiled stent framework may be coaxially located within a stent-receiving tube (not shown) when axially elongated within the stent-receiving tube, and recoiled when delivered from the stent-receiving tube and deployed adjacent to the aneurysm. For example, when placed in a stent-receiving tube of a catheter, coiled stent 220 is extended axially and contained within a thin-walled tube with an inner diameter sized slightly larger than the diameter of the wire for later deployment. In another example, coiled stent 220 is compressed into the stent-receiving tube and covered with a thin sheath that retracts to allow deployment of the stent.

In other embodiments, the inner and outer diameter of coiled stent 220 may vary along the axial length of the stent to aid in securing the stent within an endovascular vessel while minimizing the outward forces along the vessel and to aid in occluding the aneurysm. The diameter and cross-sectional geometry of the stent framework wire may be varied along the coil windings, and the density of windings per unit length may be varied along the axial length to provide tailored stiffness of coiled stent 220, as well as to provide a tighter web along at least a portion of coiled stent 220, which helps occlude an aneurysmal neck or mouth of an aneurysmal sack.

Coiled stent 220 may comprise a metallic base. The metallic base may comprise a superelastic metal such as nitinol, a nickel-tin alloy, a shape-memory material, and other superelastic materials. These materials are preferred particularly because they can be formed into the desired final shape, undergo large deformations by stretching, pulling or bending, and returned to the desired final shape when no longer loaded or constrained. With elastic strain limits of up to 8% or more for superelastic materials, significant distortions of the superelastic material can be made without permanently or plastically deforming the stent. Small diameter wire of superelastic material formed into a tightly wound helical coil, for example, can be significantly deformed by axially extending it into a straight wire by pulling, and then returned to its tightly wound configuration after release. Since the delicate vasculatures of the brain have relatively smaller diameters than other vessels in the body, the diameter of the coiled stent, the diameter of the stent framework wire, and the deployment mechanism also need to be small.

The stent framework wire can be formed from materials that are not superelastic, because bending strains induced in the stent framework wire when the coiled stent is axially extended are usually small when the wire diameter is small and the diameter of the coiled stent is sufficiently large. The stent framework wire of the coiled stent may comprise a metallic base such as, for example, stainless steel, nitinol, tantalum, MP35N alloy, platinum, titanium, a chromium-based alloy, a suitable biocompatible alloy, a suitable biocompatible polymer, or combinations thereof.

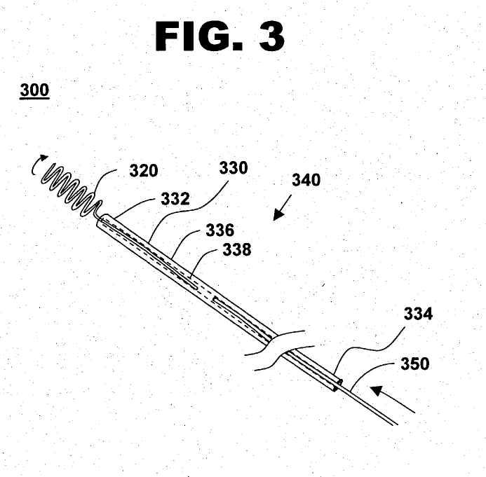

FIG. 3 shows an illustration of a system for treating an aneurysm, in accordance with one embodiment of the present invention at 300. Aneurysm treatment system 300 includes a catheter 340 with a stent-receiving tube 330, and a coiled stent 320 axially received in stent-receiving tube 330. Coiled stent 320 is delivered to the aneurysm through a vessel in the body via the catheter. Typically a catheter is inserted into a small incision, for example, in the upper thigh and guided through the bloodstream to the site of an aneurysm in the brain. Catheter 340 may include other structures for guiding catheter 340 such as a guide wire, not shown here for clarity.

Stent-receiving tube 330 has a distal end 332 and a proximal end 334. Stent-receiving tube 330 has an outer diameter 336 and an inner diameter 338. Outer diameter 336 and inner diameter 338 may be sized to contain axially-received coiled stent 320. In one embodiment, stent-receiving tube 330 comprises a thin-walled tube with inner diameter 338 sized to contain axially-received coiled stent 320. Stent-receiving tube 330 comprises part of a catheter that houses a guide wire and other facilities for positioning and deploying the axially-received coiled stent.

Stent-receiving tube 330 may comprise a thin-walled tube with an inner diameter sized to receive axially expanded coiled stent 320 and any coatings on the coiled stent framework. In another embodiment, stent-receiving tube 330 includes a delivery sheath that surrounds axially-received coiled stent 320, which is torn away or retracted to allow the deployment of the stent. The delivery sheath may have, for example, an inside diameter slightly greater than the wire diameter of the coil. The catheter with the stent-receiving tube and the axially-received coiled stent provide a very low profile, flexible and deliverable implant.

Catheter 340 with stent-receiving tube 330 is inserted into a vessel by threading distal end 332 through one or more endovascular vessels in the leg, chest, neck or head to position coiled stent 320 adjacent to the aneurysm. In one embodiment, a push wire 350 is inserted into the catheter for moving axially-received coiled stent 320 through stent-receiving tube 330 and out distal end 332 of stent-receiving tube 330. As axially-received coiled stent 320 is deployed from stent-receiving tube 330, coiled stent 320 recoils back into a predetermined coiled shape. The predetermined coil shape may be, for example, a helical coil with a constant outside diameter along an axial length. Coiled stent 320 forms a helical spring when coiled stent 320 is deployed at the aneurysm. The predetermined coil shape may be, for example, a helical coil with one or more tapers or undulations along an axial length when delivered to the aneurysm.

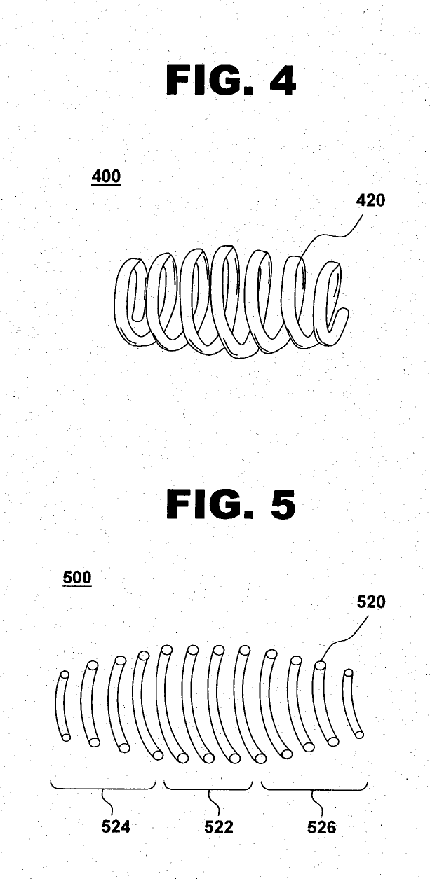

FIG. 4 shows a perspective view of a coiled stent formed as a helical spring with at least one taper or undulation, in accordance with one embodiment of the present invention at 400. Coiled stent 420 forms a helical spring with at least one taper or undulation along an axial length of the helical spring when the coiled stent is deployed at the aneurysm. In this embodiment, a linear taper begins midway along the length of coiled stent 420, reducing in diameter a uniform amount towards each end of coiled stent 420. The linear taper may aid in deployment and securing of coiled stent 420 within an intracranial vessel. In some cases, the enlarged central portion of coiled stent 420 may partially encroach into an aneurysmal sack, occluding the neck or opening of the sack, and securing coiled stent 420 from moving up and down the intracranial vessel as a result of motion or pulsations from cardiovascular activity such as blood flow.

Coiled stent 420 may have linear or curved tapers, a single taper that begins at one end and ends at the other, an inverse taper that is larger at the ends, or a combination thereof. The tapers and undulations may have an increased or decreased density of coil windings where the undulations are largest, tailored to provide desired occlusion, deployment and compliance characteristics.

FIG. 5 shows a cross-sectional view of a coiled stent formed as a helical spring with at least one taper or undulation, in accordance with another embodiment of the present invention at 500. Coiled stent 520 forms a helical spring with at least one taper or undulation along an axial length of the helical spring when the coiled stent is deployed at the aneurysm. In this embodiment, coiled stent 520 includes a central section 522 with a nominally uniform diameter, and two end sections 524, 526 extending from central section 522 towards each end of coiled stent 520 with linear tapers where the diameter of the coiled stent decreases a uniform amount. The constant diameter of central section 522 aids in occluding the neck or opening of an aneurysmal sack when deployed, preventing or limiting the flow of endovascular fluids into and out of the aneurysmal sack, thereby limiting the pressure on the aneurysmal walls and reducing the likelihood of further enlargements of the aneurysmal sack or rupture of the aneurysmal walls. The linear tapers may aid in deployment and securing of coiled stent 520 within the intracranial vessel.

The tapered regions of each end section 524, 526 of coiled stent 520 may be linear or curved. A single taper may be used at one end or the other, and an inverse taper may be used that is larger at the ends. Undulations and other sinuous variations along the length of coiled stent 520 may be included to provide increased blockage of a mouth or neck of an aneurysm. The tapers and undulations may have an increased density of coil windings, for example, at central section 522 where the diameter is nominally uniform to provide a partial webbing for limiting the transport of endovascular fluid through the sides of coiled stent 520 adjacent to the aneurysm. The density of coil windings at the central section of coiled stent 520 may be tightly wound so that adjacent coil windings form a continuous web along at least a portion of coiled stent 520. The diameter and cross-sectional geometry of the stent framework may be altered in the central region with, for example, a larger diameter wire or a rectangular winding cross section to form the web. The addition of polymer coatings or other materials onto the windings along portions of the coiled stent may also aid in the formation of a partial or a continuous web.

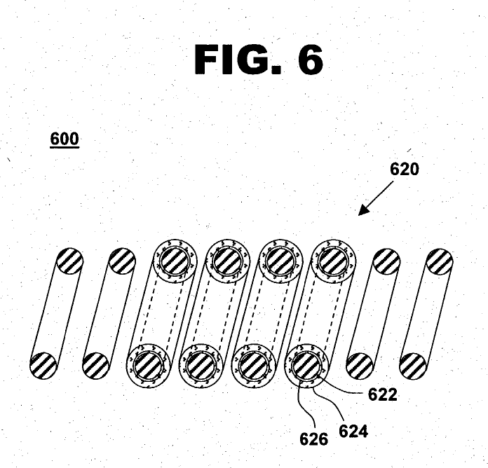

FIG. 6 shows a cross-sectional view of a coiled stent with an adhesion layer and a polymeric coating circumferentially disposed along a portion of the coiled stent, in accordance with one embodiment of the present invention at 600. Coiled stent 620 includes a coiled stent framework 622 and a polymeric coating 624 circumferentially disposed on at least a portion of coiled stent 620. An adhesion layer 626 may be positioned between coiled stent framework 622 and polymeric coating 624.

Polymeric coating 624 may be disposed along at least a portion of the length of coiled stent 620 when coiled stent 620 is deployed, such that disposed polymer coating 624 forms a partial or a continuous web along at least a portion of coiled stent 620. The thickness and number of layers of polymeric coating 624 may be determined based on the spacing between adjacent windings of coiled stent 620, such that polymeric coating 624 and coiled stent framework 622 combine to form a partial or continuous web. The partial or continuous web may be positioned adjacent to the opening or neck of an aneurysm to occlude the aneurysm, reducing the potential for breakage of the aneurysmal walls. Polymeric coated 624 may be selected in part to control the coefficient of friction between the elongated stent and a delivery tube or catheter encompassing coiled stent 620.

Polymeric coating 624 may comprise one or more polymeric materials suitable for coating coiled stent 620 and for deployment within the body. Polymeric coating 624 may comprise a biodegradable polymer or a biostable polymer. Polymeric coating 624 may comprise, for example, a biodegradable polymer such as polycaprolactone (PCL), polyglycolide (PGA) or poly(lactide-co-glycolide) (PLGA), or a biostable polymer such as a silicone-urethane copolymer, a polyurethane, or ethylene vinyl acetate (EVA).

Polymeric coating 624 may comprise, for example, a drug polymer that contains one or more bioactive agents to provide a therapeutic effect when deployed at the aneurysm. Polymeric coating 624 may contain one or more therapeutic compounds such as pharmaceutical drugs or bioactive agents. Polymeric coating 624 may contain, for example, a polymeric matrix in which one or more bioactive agents are interdispersed. One or more layers of polymeric coatings 624 may be disposed on coiled stent framework 622.

The therapeutic compounds provide treatment or prevention of one or more conditions including coronary restenosis, cardiovascular restenosis, angiographic restenosis, arteriosclerosis, hyperplasia, and other diseases and conditions. For example, a therapeutic compound can be incorporated into polymeric coating 624 to inhibit or prevent vascular restenosis, a condition corresponding to a narrowing or constriction of the diameter of the bodily lumen where the stent is placed. In one embodiment, the bioactive agent comprises an antirestenotic agent. In another embodiment, the therapeutic compound comprises a bioactive agent such as an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, or a saccharide derivative. In another embodiment, polymeric coating 624 includes a combination of pharmaceutical drugs.

A number of pharmaceutical drugs have the potential to be used in polymeric coating 624. For example, an antirestenotic agent such as rapamycin, a rapamycin analogue, or a rapamycin derivative prevents or reduces the recurrence of narrowing and blockage of the bodily vessel. An antisense drug works at the genetic level to interrupt the process by which disease-causing proteins are produced. An antineoplastic agent is typically used to prevent, kill, or block the growth and spread of cancer cells in the vicinity of the stent. An antiproliferative agent may prevent or stop targeted cells or cell types from growing. An antithrombogenic agent actively retards blood clot formation. An anticoagulant often delays or prevents blood coagulation with anticoagulant therapy, using compounds such as heparin and coumarins. An antiplatelet agent may be used to act upon blood platelets, inhibiting their function in blood coagulation. An antibiotic is frequently employed to kill or inhibit the growth of microorganisms and to combat disease and infection. An anti-inflammatory agent such as dexamethasone can be used to counteract or reduce inflammation in the vicinity of the stent. At times, a steroid is used to reduce scar tissue in proximity to an implanted stent. A gene therapy agent may be capable of changing the expression of a person's genes to treat, cure or ultimately prevent disease.

By definition, a bioactive agent is any therapeutic substance that provides prevention or treatment of disease or disorders. An organic drug is any small-molecule therapeutic material. A pharmaceutical compound is any compound that provides a therapeutic effect. A recombinant DNA product or a recombinant RNA product includes altered DNA or RNA genetic material. Bioactive agents of pharmaceutical value may also include collagen and other proteins, saccharides, and their derivatives.

Polymeric coating 624 elutes at least one bioactive agent. Polymeric coating 624 may include and elute multiple bioactive agents. Polymeric coating 624 can be tailored to control the elution of one or more bioactive agents primarily by diffusion processes. In some cases, a portion of the polymeric coating is absorbed into the body to release bioactive agents from within the coating.

Incorporation of a drug polymer into polymeric coating 624 allows, for example, the rapid delivery of a pharmacologically active drug or bioactive agent within twenty-four hours of surgery, with a slower, steady delivery of a second bioactive agent over the next three to six months.

Polymeric coating 624 may include a plurality of drugs, each drug having a predetermined elution rate. In one embodiment, a first bioactive agent is concentrated adjacent to the stent framework, and a second bioactive agent is concentrated adjacent to the outer surface of polymeric coating 624. For example, the first bioactive agent may comprise an antirestenotic drug such as rapamycin, a rapamycin analogue, or a rapamycin derivative. The second bioactive agent may comprise an anti-inflammatory drug such as dexamethosone.

Adhesion layer 626 may be positioned between polymeric coating 624 and coiled stent framework 622 to improve the adhesion of the polymeric coating and its durability. Adhesion layer 626 may be a polymeric material or any material that adheres well to the underlying stent framework, particularly a metallic base of coiled stent 620. Adhesion layer 626 is selected to adhere well to coiled stent framework 622 and to be readily coated with another polymeric material such as polymeric coating 624. Adhesion layer 626 may be any suitable adhesion layer material such as parylene, polyurethane, phenoxy, epoxy, polyimide, polysulfone, or pellathane.

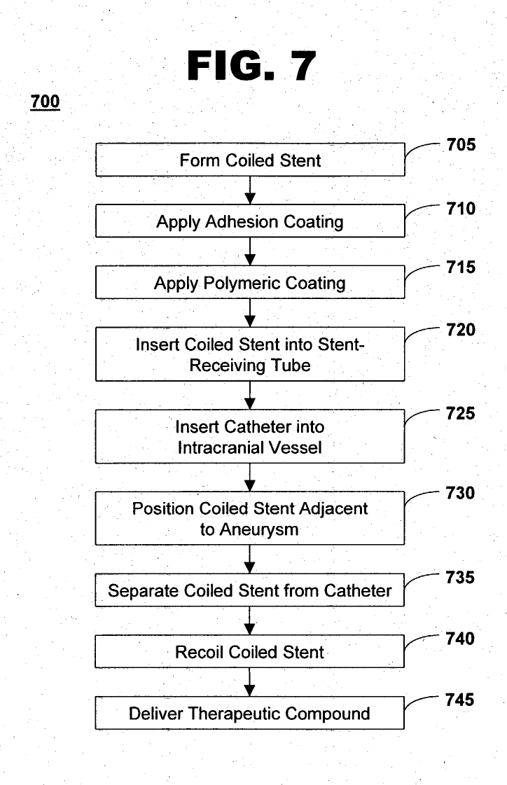

FIG. 7 shows a flow diagram of a method of treating an intracranial aneurysm, in accordance with one embodiment of the present invention at 700. Intracranial aneurysm treatment method 700 comprises steps to form, position, deploy and treat an intracranial aneurysm including axial aneurysms, lateral aneurysms, saccular or berry aneurysms, and other aneurysms within the cranial vasculature, and to treat different types of aneurysms in vessels elsewhere in the body.

A coiled stent is formed, as seen at block 705. The framework of the coiled stent may comprise a metallic base. The coiled stent may be formed from superelastic materials such as nitinol, a nickel-tin alloy, or a shape-memory material. The coiled stent may be formed, for example, from a piece of small-diameter nitinol wire that is wound around a form and heat-treated or shape-set above the martensite-austenite phase transition temperature to retain the wound shape. Temperatures between, for example, 490 degrees centigrade and 525 degrees centigrade and higher may be used to heat-treat and shape set the coiled stent.

For certain shape-memory alloys, the transition temperature may be below body temperature and below room temperature. For coiled stents using these alloys, the coil may be chilled, straightened or axially elongated, and inserted into the stent-receiving tube. When delivered to the aneurysm, the axially-elongated coiled stent would heat up above the transition temperature and return to its helically coiled form. When loaded into a sufficiently robust stent-receiving tube or a delivery catheter sheath, the axially elongated coiled stent may be chilled, axially elongated, and inserted into the stent-receiving tube where the coiled stent warms up and transitions to the austenite phase transition temperature while retained in the elongated shape until released from the stent-receiving tube.

Alternatively, the transition temperature of the shape-memory alloy may be above room temperature and below normal body temperature, such that deformations of the coiled stent at and below room temperature quickly recover when the coiled stent is deployed and heated to body temperature. In another embodiment, the transition temperature of the shape-memory alloy is set above normal body temperature. The superelastic properties of shape-memory material in the martensite phase such as high elastic strain limits allow the coiled stent to be longitudinally expanded into a small-diameter receiving tube for catheter deployment, while readily restoring the coiled stent to a helical shape when deployed and separated from the receiving tube. A thin-walled stent-receiving tube or a sheath on a delivery catheter constrains the elastically elongated coiled stent until deployed.

With fine enough wire, the coiled stent may be made from other non-superelastic, non-shape memory materials. The coiled stent may be formed from other materials such as stainless steel, nitinol, tantalum, MP35N alloy, platinum, titanium, a chromium-based alloy, a suitable biocompatible alloy, a suitable biocompatible polymer, or a combination thereof. Particularly in cases where the coiled stent framework comprises small diameter wire and the outer diameter of the coiled stent is not excessively small, these materials are suitable because the strain induced when axially elongated does not exceed the elastic strain limit of the material used. In one embodiment, a wire of one of these materials is wound around a form or fixture to establish the desired shape of the coiled stent, and then the material is heat-treated and annealed to reduce stresses in the material, locking in the preferred shape. In another embodiment, a wire of one of these materials is pulled across a small radius fixture and then heat-treated to retain the coiled shape. Alternately, the wire is annealed and thermally softened, then wound around a small-diameter form to plastically deform the wire into the desired shape. After allowing an amount of elastic recoil, the coil windings may then be swaged or cold-worked to increase the yield stress and improve the range of elastic behavior.

The coiled stent may be shape-set to have a small spacing between adjacent turns of the coil. The coiled stent may be shape-set to form a helical spring with a uniform outer diameter or with one or more tapers or undulations.

Alternatively, the coiled stent may be formed from wire with a variable diameter along its length to provide variable strength along the length. Very fine-diameter wire with a tailored radial strength may be used to form the coiled stent for deployment in the delicate vasculature of the brain. In another embodiment, the coiled stent framework comprises an elongated or a rectangular cross section.

An adhesion layer may be disposed on the metallic base of the coiled stent, as seen at block 710. The adhesion layer may be disposed on a metal wire prior to winding the coil. Alternatively, the adhesion layer may be disposed on at least a portion of the coiled stent framework after the coiled stent has been wound. The adhesion layer may be applied using any suitable technique such as dipping, painting, brushing or spraying, then dried in a vacuum or controlled environment at room temperature or an elevated temperature. The adhesion layer may be positioned between the metallic base of the coiled stent and a polymeric coating to help adhere the polymeric coating to the metallic stent framework.

A polymeric coating may be circumferentially disposed on at least a portion of the coiled stent, as seen at block 715. The wire forming the coiled stent framework may be coated, for example, about its circumference with a biocompatible polymer. When recoiled and deployed, the coating can partially or completely seal the space between adjacent turns of the coiled stent preventing leaks to the aneurysmal sack.

The polymeric coating may be applied by any suitable technique such as dipping, painting, brushing or spraying, and then dried by driving off any solvents and cured as needed. The polymeric coating may be applied when the coiled stent has been axially expanded, or when formed or recoiled into the desired helical shape. The polymeric coating may be applied onto an adhesion layer that is disposed at least a portion of the coiled stent to improve adhesion between the polymeric coating and the coiled stent framework.

The polymeric coating may be applied to a suitable thickness using one or more polymeric layers such that the disposed polymeric coating forms a continuous or nearly continuous web along at least a portion of the coiled stent when the coiled stent is deployed.

The polymeric coating disposed on the coiled stent framework may comprise a drug polymer. One or more bioactive agents may be interdispersed throughout the polymeric coating for delivery when deployed at an aneurysm. One or more layers of drug polymers may be applied to the coiled stent framework to control the elution rate of one or more drugs included in the polymeric coating. One or more barrier layers may be included between the drug polymer layers or on the perimeter of the polymeric coating to control the elution rate of the pharmaceutical compounds.

The coiled stent is inserted into a stent-receiving tube, as seen at block 720. The stent-receiving tube comprises part of a catheter that houses a guide wire and other facilities for positioning and deploying the axially-received coiled stent. The stent-receiving tube may comprise a thin-walled tube with an inner diameter sized to receive an axially expanded coiled stent and any coatings on the coiled stent framework. Alternatively, the stent-receiving tube includes a delivery sheath that surrounds the axially-received coiled stent, which is torn away or retracted to allow the deployment of the stent. The delivery sheath may have an inside diameter slightly greater than the wire diameter of the coil. The coiled stent is sterilized typically prior to insertion into the stent-receiving tube.

The catheter including an axially-received coiled stent is inserted into a vessel in the body, as seen at block 725. The catheter may be inserted, for example, through one or more vessels in the head, neck, chest or leg. The vessel may be located, for example, in the intracranial vasculature of the body. The catheter with the axially-received coiled stent is inserted into an intracranial vessel through a suitable entry point for the endovascular procedure.

The axially-received coiled stent may be positioned adjacent to the intracranial aneurysm, as seen at block 730. The axially-received coiled stent may be positioned by physically maneuvering the tip of the catheter with a guide wire through a sometimes tortuous path within the head and neck. Any suitable imaging system such as x-ray and fluoroscopic imaging systems may be used to determine the position of the catheter tip and guide it towards the aneurysm. For example, a radio-opaque marker may be attached to the end of the catheter or to one or more locations along the coiled stent.

The axially-received coiled stent may be separated from the catheter, as seen at block 735. In one example, the axially-received coiled stent is separated from the catheter by retracting a delivery sheath from around the axially-received coiled stent and deploying the coiled stent adjacent to the aneurysm. In another example, the axially-received coiled stent is separated from the catheter by use of a push wire for moving the axially-received coiled stent through the stent-receiving tube and deploying the coiled stent adjacent to the aneurysm. The push wire may be operated, for example, by gripping a proximal end of the push wire and gently pushing the axially-received coiled stent out the distal end of the stent-receiving tube to separate the coiled stent from the stent-receiving tube. As the axially-received coiled stent emerges from the distal end of the stent-receiving tube, it reforms or recoils into a helical shape when delivered from the stent-receiving tube and deployed adjacent to the aneurysm.

When the coiled stent is separated from the stent-receiving tube, the stent recoils, as seen at block 740. The stent recoils into a helical shape that may include one or more tapers or undulations. When the coiled stent is deployed, at least a portion of the recoiled stent occludes an aneurysmal neck or opening of the intracranial aneurysm. The aneurysmal neck may be completely or partially occluded, for example, by placing a portion of the coiled stent adjacent to the aneurysm. The coiled stent may include tightly spaced windings in the region adjacent to the aneurysm. The coiled stent may include a polymeric coating over the windings such that a continuous web is formed along at least a portion of the coiled stent when the coiled stent is deployed.

A therapeutic compound may be delivered to the intracranial vessel or the intracranial aneurysm with one or more pharmaceutical drugs or bioactive agents interdispersed within a polymeric coating, as seen at block 745. A polymeric coating with one or more interdispersed therapeutic compounds may be circumferentially disposed on at least a portion of the coiled stent when the coiled stent is deployed. The therapeutic compounds may be delivered to the aneurysm or the vessel or both over a period of minutes, hours, days, or even months, depending on the bioactive agent and the elution characteristics of the polymeric coating and any barrier coatings disposed thereon.

While described in the confines of a method of treating an intracranial aneurysm, the method can be applied to any aneurysm or vessel in another part of the body where an axially-received coiled stent can be deployed. Other surgical procedures such as placing tiny vaso-occlusive coils within the aneurysmal sack can be delivered prior to and in combination with the axially-received coiled stent, such that the coiled stent aids in retaining any vaso-occlusive coils within the aneurysmal sack when occluding the neck or opening of the aneurysm. In one embodiment of the present invention, vaso-occlusive coils may be attached to the coiled stent framework to further aid in retention and placement of the vaso-occlusive coils in the aneurysmal sack.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

I claim:

1. A system for treating an aneurysm, comprising:
a catheter including a stent-receiving tube; and
a coiled stent axially received in the stent-receiving tube, wherein the coiled stent is delivered to the aneurysm through a vessel via the catheter.

2. The system of claim 1 wherein the catheter includes a delivery sheath that retracts to deploy the stent.

3. The system of claim 1 wherein the catheter includes a thin-walled tube with an inner diameter sized to contain the axially-received coiled stent.

4. The system of claim 1 wherein the coiled stent forms a helical spring when the coiled stent is deployed at the aneurysm.

5. The system of claim 1 wherein the coiled stent forms a helical spring with at least one taper or undulation along an axial length of the helical spring when the coiled stent is deployed at the aneurysm.

6. The system of claim 1 wherein the coiled stent comprises a metallic base.

7. The system of claim 6 wherein the metallic base comprises a superelastic metal selected from the group consisting of nitinol, a nickel-tin alloy and a shape-memory material.

8. The system of claim 6 wherein the metallic base comprises a material selected from the group consisting of stainless steel, tantalum, MP35N alloy, platinum, titanium, a chromium-based alloy, a suitable biocompatible alloy, a suitable biocompatible polymer, and a combination thereof.

9. The system of claim 1 further comprising:
a push wire for moving the axially-received coiled stent through the stent-receiving tube and deploying the coiled stent adjacent to the aneurysm.

10. The system of claim 1 further comprising:
a polymeric coating circumferentially disposed on at least a portion of the coiled stent.

11. The system of claim 10 wherein the disposed polymeric coating forms a continuous web along at least a portion of the coiled stent when the coiled stent is deployed.

12. The system of claim 10 wherein the disposed polymeric coating comprises a drug polymer.

13. The system of claim 10 further comprising:
an adhesion layer positioned between the disposed polymeric coating and a metallic base of the coiled stent.

14. A coiled stent for treating an aneurysm, comprising:
a coiled stent framework, the coiled stent framework coaxially located within a stent-receiving tube when axially elongated within the stent-receiving tube, and recoiled when delivered from the stent-receiving tube and deployed adjacent to the aneurysm.

15. The coiled stent of claim 14 wherein the coiled stent forms a helical spring when the coiled stent is deployed at the aneurysm.

16. The coiled stent of claim 14 wherein the coiled stent forms a helical spring with at least one taper or undulation along an axial length of the helical spring when the coiled stent is deployed at the aneurysm.

17. The coiled stent of claim 14 wherein the coiled stent comprises a metallic base.

18. The coiled stent of claim 17 wherein the metallic base comprises a superelastic metal selected from the group consisting of nitinol, a nickel-tin alloy and a shape-memory material.

19. The coiled stent of claim 17 wherein the metallic base comprises a material selected from the group consisting of stainless steel, tantalum, MP35N alloy, platinum, titanium, a chromium-based alloy, a suitable biocompatible alloy, a suitable biocompatible polymer, and a combination thereof.

20. The coiled stent of claim 14 further comprising:
a polymeric coating circumferentially disposed on at least a portion of the coiled stent.

21. The coiled stent of claim 20 wherein the disposed polymer coating forms a continuous web along at least a portion of the coiled stent when the coiled stent is deployed.

22. The coiled stent of claim 20 wherein the disposed polymeric coating comprises a drug polymer.

23. The coiled stent of claim 14 further comprising:
an adhesion layer positioned between the disposed polymeric coating and a metallic base of the coiled stent.

24. A method of treating an intracranial aneurysm, comprising:
inserting a catheter including an axially-received coiled stent into a vessel in a body;
positioning the axially-received coiled stent adjacent to the intracranial aneurysm;
separating the axially-received coiled stent from the catheter; and
recoiling the axially-received coiled stent; wherein at least a portion of the recoiled stent occludes an aneurysmal neck of the intracranial aneurysm when the coiled stent is deployed.

25. The system of claim 24 wherein the aneurysm comprises an axial aneurysm, a lateral aneurysm, or a saccular aneurysm.

26. The system of claim 24 wherein the vessel is located in an intracranial vasculature of the body.

27. The method of claim 24 further comprising:
delivering a therapeutic compound to the intracranial vessel or the intracranial aneurysm with a polymeric coating circumferentially disposed on at least a portion of the coiled stent when the coiled stent is deployed, the polymeric coating including at least one therapeutic compound.
